(19) United States
(12) Patent Application Publication
(10) Pub. No.: US 2014/0121654 A1
(43) Pub. Date: May 1, 2014

(54) SURGICAL LASER SETUP CALCULATIONS INTERFACE FOR CORNEAL MULTI-PLANE INCISION

(71) Applicant: James Carlton Loden, Nashville, TN (US)

(72) Inventor: James Carlton Loden, Nashville, TN (US)

(21) Appl. No.: 13/663,330

(22) Filed: Oct. 29, 2012

(51) Int. Cl. A61B 18/20; A61F 9/01

(52) U.S. Cl. 606/6; 606/13; 606/4

(57) ABSTRACT

An apparatus and a method are described for setting up a medical laser machine configured to be used in eye surgery for making a multi-planar incision. In various embodiments, a software interface is provided to receive, from a user, one or more dimensional measurements of an eye of a patient as input, automatically calculate precise geometric and other setup parameters, based on a set of equations, of a multi-planar incision for the patient's eye, and provide the calculated parameters to the user for setting up the medical laser machine to make the incision.

### CROSS-REFERENCE(S) TO RELATED APPLICATION(S)

This application is related to U.S. application Ser. No. 13/267,485, filed on 6 Oct. 2011, the disclosure of which is hereby expressly incorporated by reference in its entirety.

### TECHNICAL FIELD

This application relates generally to eye surgery and more particularly to systems, devices and methods for calculating setup parameters for a medical laser machine for a corneal incision.

### BRIEF DESCRIPTION OF THE DRAWINGS

The drawings, when considered in connection with the following description, are presented for the purpose of facilitating an understanding of the subject matter sought to be protected.

FIG. 1A shows an example diagram of a human eye structure showing its main constituents;

FIG. 1B shows an example software interface for setting up a medical laser configured to be used in eye surgery;

FIG. 2 shows an example human eye diagram with a clouded lens due to cataract disease;

FIG. 3 shows an example human eye diagram with surgical instruments inserted inside the corneal anterior chamber during surgery;

FIG. 4A shows an example software interface diagram configured to be used to specify an angle of corneal incision;

FIGS. 4B-4D show examples of software interfaces configured to be used to specify an angular span over which a medical laser machine is active during corneal incision;

FIG. 4E shows an example spiral cutting path of a laser beam;

FIG. 5 is an example diagram of a cornea of a human eye showing a location of a corneal incision;

FIG. 6A is an example diagram of a Detail A of the cornea of FIG. 5 showing a detailed structure of an incision in the cornea; and

FIG. 6B is an example diagram of the Detail A of the cornea of FIG. 5 showing the top view corresponding to the side view shown in FIG. 6A of the incision in the cornea.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while following description references corneal incision, it will be appreciated that the disclosure may be used with other types of laser surgery.

Briefly described, an apparatus and a method are described for setting up a medical laser machine configured to be used in eye surgery for making a multi planar incision. In various embodiments, a software interface is provided to receive, from a user, one or more dimensional measurements of an eye of a patient as input, automatically calculate precise geometric and other setup parameters, based on a set of equations, of a multi planar incision for the patient's eye, and provide the calculated parameters to the user for setting up the medical laser machine to make the incision.

Medical lasers have been gaining in popularity in various surgical treatments recently because of faster patient recovery times, more efficient surgical procedure, higher precision, less physical intrusion, and less bleeding. One of the surgical procedures for which lasers may be used is cataract surgery.

Cataract surgery generally removes a clouded or damaged lens from a patient's eye. A new lens may then be inserted into the eye to correct the patient's vision. All conventional types of cataract surgery require one or more incisions in the cornea. In many applications, a clear corneal incision includes a small incision approximately in the plane of the cornea ranging from about 1.8 to about 3.5 mm in width. The incision passes completely through the cornea at an incision site near the limbus, forming a tunnel through which a surgeon may insert a tool for lens removal, such as a phacoemulsification tool. The tool may be used to break up and remove the opaque or clouded lens. Such clear corneal incisions for cataract surgery are conventionally formed using a small blade known as a keratome. The keratome, which is made of steel or diamond material, includes a blade width and shape substantially equal to the size and shape of the desired clear corneal incision. Traditionally, the keratome is manually inserted directly into the cornea by the surgeon to form corneal incision.

Conventional tools and free-hand methods for forming corneal incisions during cataract surgery using a keratome can cause abnormally large incisions, improper wound architecture leading to non-sealing leaky wounds, and descemet's membrane detachments and trauma to the eye. Such trauma may result in improper postoperative sealing along the incision site.

Such complications may be reduced by using keratomes and associated incision procedures to produce three-dimensional incisions. For example, some conventional keratomes and associated incision methods may be used to produce a two-step or a three-step planed incision through the cornea. Such three-dimensional incisions may reduce postoperative complications such as inadequate sealing or extended healing times. However, such devices and methods still utilize a mechanical blade that involves mechanical contact to the cornea and surrounding tissues. Such mechanical contact makes further complications possible, including improper sealing, tearing and distortion. As a result, conventional clear corneal incisions may require suturing or extended recovery times.

Incisions for ophthalmic surgery may also be performed in other types of surgeries using a laser instead of a mechanical keratome. For example, lasers may be used in laser-assisted in situ keratomileusis, or LASIK eye surgery, for correcting such refractive conditions as myopia, hyperopia or astigmatism. During a LASIK procedure, a corneal flap is created by forming a hinged, ring-shaped incision in the cornea. The corneal flap incision may be formed using a femtosecond laser rather than a conventional keratome. During flap creation, the laser is focused at a subsurface region of the cornea. The laser is pulsed at a predetermined frequency, and each series of pulses creates a small subsurface bubble in the cornea via Intrastromal (within foundation of an organ), Ablation (surgical removal), or ISA. Multiple bubbles may be formed locally in a ring shape to provide a corneal flap that can be lifted by the surgeon for accessing the underlying corneal stroma. The corneal stroma may then be reshaped using an excimer laser. Following the surgery, the corneal flap may be closed.

LASIK eye surgery using a femtosecond laser system has recently become a common medical procedure. As such, femtosecond laser systems for LASIK eye surgery are commonly owned by eye surgeons. However, conventional ophthalmic surgery femtosecond laser systems designed for LASIK are generally configured for making a hinged, round flap-style incision for revealing the corneal stroma, and are not configured for producing a clear corneal incision of the type needed for cataract surgery.

Because so many femtosecond laser systems for LASIK eye surgery are already in use, it would be beneficial if such devices were also configurable for performing both LASIK eye surgery, which requires a hinged, round flap incision, and cataract surgery, which requires a small clear corneal incision extending completely through the cornea.

A lens cover or mask, in the form of a small disk with a radial slot, as described in detail in the related U.S. patent application Ser. No. 13/267,485, incorporated herein by reference, provides a device and associated methods that allow a conventional ophthalmic femtosecond surgical laser system to be used to make a clear corneal incision for cataract surgery. Conventionally, such femtosecond surgical laser systems have been used to form a circular, corneal flap for LASIK eye surgery. However, by partially blocking some of the incident laser light, or incident laser beam, emitted from the femtosecond surgical laser system using the lens cover, such conventional laser systems may be used in additional applications requiring a smaller incision that extends completely through the cornea, such as a clear corneal incision for insertion of a phacoemulsification device for removing a cataracted lens. The laser beam passes through the slot in the lens cover to make an incision in the cornea while the beam is blocked by the non-slotted portions of the cover.

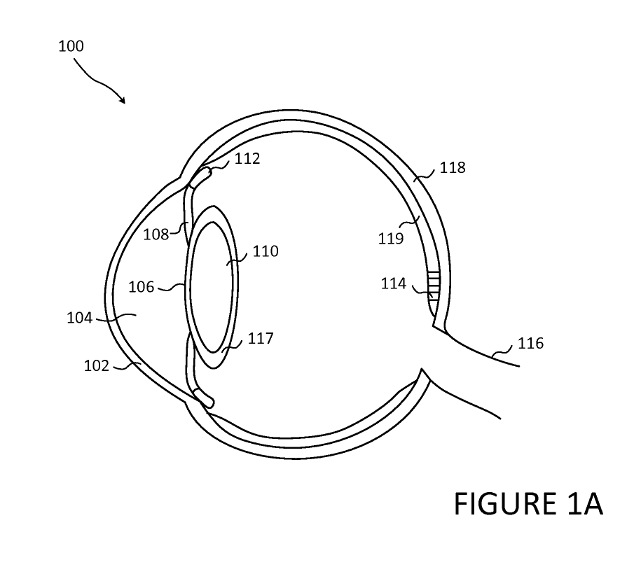

FIG. 1A shows an example diagram of a human eye structure showing its main constituents. General eye structure 100 includes cornea 102 having anterior chamber 104 and pupil 106, which is the exposed area of lens 110 enclosed by lens bag or sack 117. Lens 110 is attached by zonules to ciliary muscles 112, while iris 108 acts as an aperture to control the amount of light reaching the pupil. The eye has a sclera layer 118, which is a fibrous protective layer encasing retina 119, the light-sensitive portion of the eye containing the rods and cones photoreceptor cells. Macula 114 is a focal area of the retina for sharp focus. Retina is connected to optic nerve 116 carrying light information to the brain for visual perception.

FIG. 1B shows an example software interface for setting up a medical laser configured to be used in eye surgery. In various embodiments, Laser Setup User Interface (LSUI) 120 is configured to allow a user, such as a physician or a medical technician, to input a patient's eye parameters or measurements and subsequently be provided with the parameters needed to set up a laser machine for making an incision in a tissue, such as eye corneal tissue, with a desired shape and dimensions. In various embodiments, LSUI is implemented as a software interface module executing on a processor in a computing device, such as a Personal Computer (PC), a dedicated controller, a networked central computer, and the like. LSUI may include an input module for receiving user inputs, a processing or calculation module for calculating setup parameters based on a set of equations, a display module for displaying status and results data, a communication module for transmitting data to and from a laser machine, and other similar software modules as needed.

Those skilled in the art will appreciate that a software interface may be implemented on a computing device which is local or remote, dedicated or general purpose, and/or stationary or mobile such as a smartphone.

Additionally, in various embodiments, other computing or processing software modules running on the same or a different computing device may be coupled with the user interface to receive and process the user inputs, calculate laser cut parameters and other laser machine setup parameters based on the user inputs and other factors like the laser machine's specifications and capabilities, and display the results via the user interface. Those skilled in the art will appreciate that various software processing functions may be performed by one or multiple cooperating software modules without departing from the spirit of the present disclosures.

In various embodiments, LSUI includes different fields to allow the user to enter data and receive laser setup parameters, calculated by the LSUI based on the user's inputs, for setting up the medical laser machine for surgery. For example, the LSUI may include a patient information section 122, a laser target section 124 having a laser cross-hair reticle 126, Corneal Depth (thickness of cornea) input field 148, and several output sections including separate sections for various cut (incision) parameters such as ring lamellar cut 128, anterior side cut 140, and posterior side cut 136.

In various embodiments, each of the output sections includes the relevant calculated parameters needed to set up the laser machine. For example, Ring Lamellar Cut section may include parameters 130 such as Depth in Cornea, Outer Diameter, Inner Diameter, (laser) Energy, Spiral Start, and the like. Each parameter may have a corresponding alphanumeric, symbolic, or other data field 132 for displaying calculated parameters. Similarly, Anterior Side Cut 140 output section may include parameters 142 such as Posterior Depth, Diameter, Energy, Cut Positions, Cut Angles, and the like. In a further similar fashion, Posterior Side Cut 136 output section may include parameters and corresponding data fields 138 such as Anterior Depth, Diameter, Energy, Cut Positions, Cut Angles, and the like.

Those skilled in the art will appreciate that different laser machines may need different setup parameters. Accordingly, LSUI may include a general setup area 144 including Options 146 for selecting a type of laser machine to be used in a surgical operation, among other setup options and preferences like color schemes, screen layout, font size, data formats, data communications, user authorization parameters, and the like.

In operation, cross hair reticle 126 may be used by the surgeon to target a particular starting point or area of a patient's body tissue, such as a small site on the eye corneal tissue, for applying the laser beam to make an incision. The laser beam may start at the starting point thus targeted by the surgeon and may create the incision in multiple, often circular traversals over the incision area, by a series of pulses each of which creates a small subsurface bubble in the cornea via intrastromal ablation. Depending on the shape and size of the cut desired, the beam traversal paths may be in the form of a spiral starting from a center and moving out with ever increasing radii. In some embodiments, throughout the path of the beam traversal the intensity of the beam may be varied under program control or the beam may be turned off and on at different points, or a combination of both.

In various embodiments, the user, for example the surgeon, may input the measured depth of the cornea of a particular patient in preparation for a surgical operation on the particular patient. The user then enters this measured value in the input field Corneal Depth 148 to start the calculation process of the laser machine's setup parameters. Once the setup parameters are computed based on the user's input, the setup parameters are displayed on the output sections, such as sections 128, 136, and 140. The user may then set up the laser machine based on these results. In some embodiments, LSUI may be coupled with the laser machine directly to automatically set up the laser machine based on the computed setup parameters.

In various embodiments, the setup parameters are computed based on the following equations:

Input or known (measured) variable: Limbal Pachy (corneal thickness for each patient)

Outputs: Calculated results according to the following equations:

I. Ring Lamellar Cut

Anterior Side Cut Change=Limbal Pachy/(Tangent(Cut Angle)); (1)

Cut Depth (in cornea)=Limbal Pachy×A; (2)

Outer Diameter=(Anterior Side Cut Diameter−(B×Anterior Side Cut Change))+C; (3)

Inner Diameter=(Outer Diameter−(B×Shelf Length))−D; (4)

II. Anterior Side Cut

Posterior Depth=(Limbal Pachy×A)+E; (5)

Cut Depth (in cornea)=Limbal Pachy×A; (6)

II. Posterior Side Cut

Anterior Depth=(Limbal Pachy×A)−E; (7)

Posterior Depth=(Limbal Pachy+F); (8)

Diameter=(Inner Diameter+D); (9)

Where: A, B, C, D, E, F, Anterior Side Cut Diameter, and Cut Angle are constants.

The above calculations are repeated for each eye of the patient. In various embodiments, the values of the constants A, B, C, D, E, and F may be set, for example, as follows: A=0.5, B=2, C=0.1, D=0.2, E=10, and F=100.

In various embodiments, equations (1)-(9) may be used to set up the laser machine's diameter of the circular traversal path for making the cut, such as the initial or inner diameter of the spiral and the final or outer diameter of the spiral. Similarly, the depth of cut and angle of cut with respect to the cornea may be calculated and set up in the laser machine.

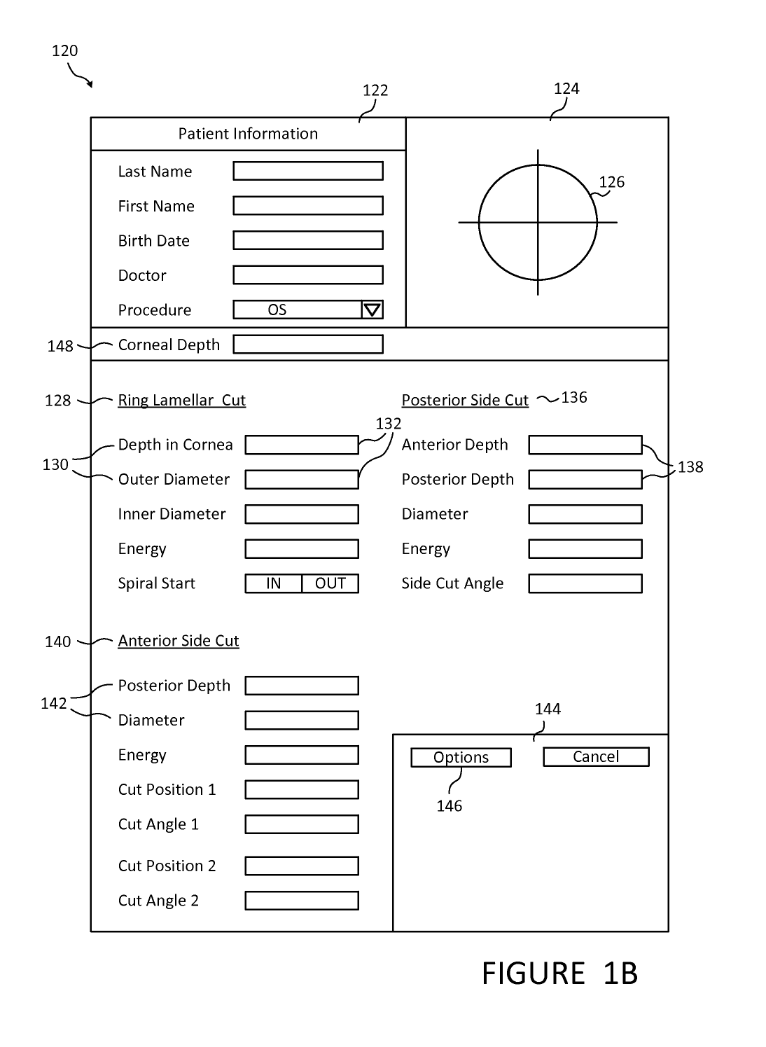

FIG. 2 shows an example human eye diagram with a clouded lens due to cataract disease. Eye structure 200 of a cataract patient includes cornea 202 and iris 208 over clouded lens 210, which may partially or completely obscures vision. When the lens is clouded, it is generally removed and replaced by cataract surgery.

Cataract surgery is employed to remove and replace (with an artificial lens) the natural or crystalline lens of the eye that has developed an opacification, referred to as cataract. Over time, physiological changes of the fibers in the crystalline lens may result in the formation of the cataract and loss of transparency in the lens, causing partial or complete impairment of vision. Symptoms may include glare from light sources in dim conditions such as nights, reduced visual acuity under dim lighting conditions, and the like.

The two main types of cataract surgery performed are phacoemulsification (“phaco”) and conventional ExtraCapsular Cataract Extraction (ECCE). In both types of surgeries an intraocular lens is inserted through a small incision in the cornea. Generally, foldable lenses may be used when phaco is performed while non-foldable lenses may be employed when ECCE is performed. The small incision size used in phacoemulsification (about 2-3 mm) may allow sutureless wound closure and healing. ECCE may utilize a larger wound (about 6-12 mm) and may require stitches to close the incision.

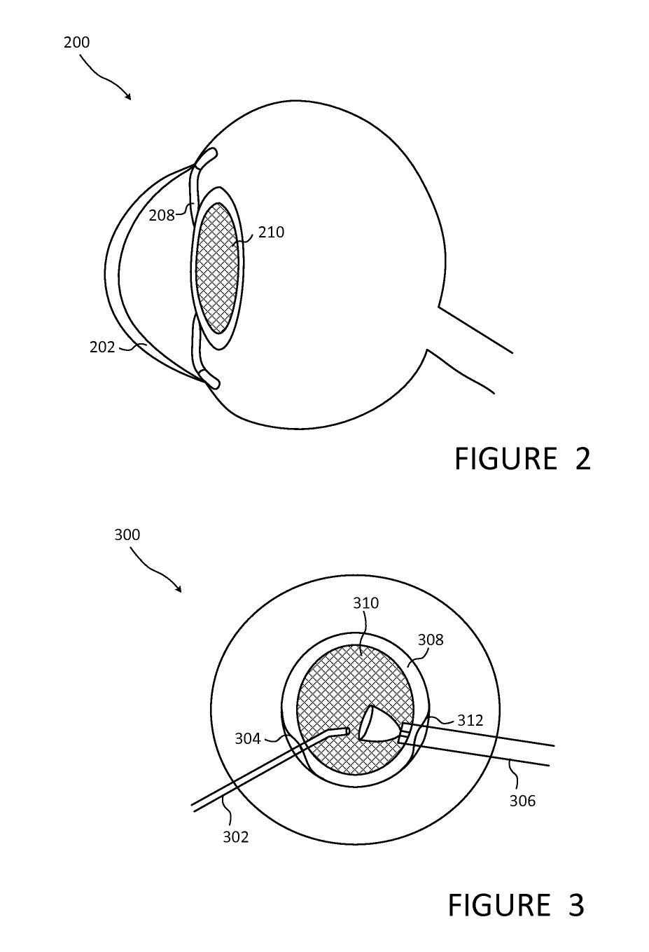

FIG. 3 shows an example human eye diagram with surgical instruments inserted inside the corneal anterior chamber during surgery. During cataract surgery, it may be desirable to form a second incision, or a second clear corneal incision, in the patient's eye. The second incision may be used for a paracentesis port for such applications as regulating the volume and pressure of fluid in the anterior chamber of the eye during removal of the cataracted lens. In other embodiments, the second incision may be used to insert a second tool such as a chopper for fragmenting the opacified/damaged lens or lens material for enhanced phacoemulsification. In some applications, it may be desirable to create the second incision in the cornea using the same femtosecond laser that is used to create the clear corneal incision. By using a laser to create the second incision, mechanical trauma to the eye associated with formation of the incision may be reduced.

In various embodiments, surgical arrangement 300 includes instruments 302 and 306 inserted into the anterior chamber of the cornea 310 through incisions 304 and 312, respectively, to access the eye lens with cataract adjacent to iris 308. In addition to the main tool for lens removal, such as phacoemulsification tool 306, passed through the first incision, a second incision may be used to insert a second tool such as chopper 302 for fragmenting the opacified/clouded lens for enhanced phacoemulsification.

In various embodiments, the removal of the lens involves the use of a machine with an ultrasonic hand piece equipped with a titanium, steel, or other suitable tip. The tip vibrates at ultrasonic frequency (for example, 40,000 Hz) and the lens material is emulsified. A second fine instrument (sometimes called a “cracker” or “chopper”) may be used from a side port to facilitate cracking or chopping of the lens nucleus into smaller pieces. Fragmentation into smaller pieces makes emulsification easier, as well as the aspiration of cortical material (soft part of the lens around the nucleus). After phacoemulsification of the lens nucleus and cortical material is completed, a dual Irrigation-Aspiration (“I-A”) probe or a bimanual I-A system is used to aspirate out the remaining peripheral cortical material.

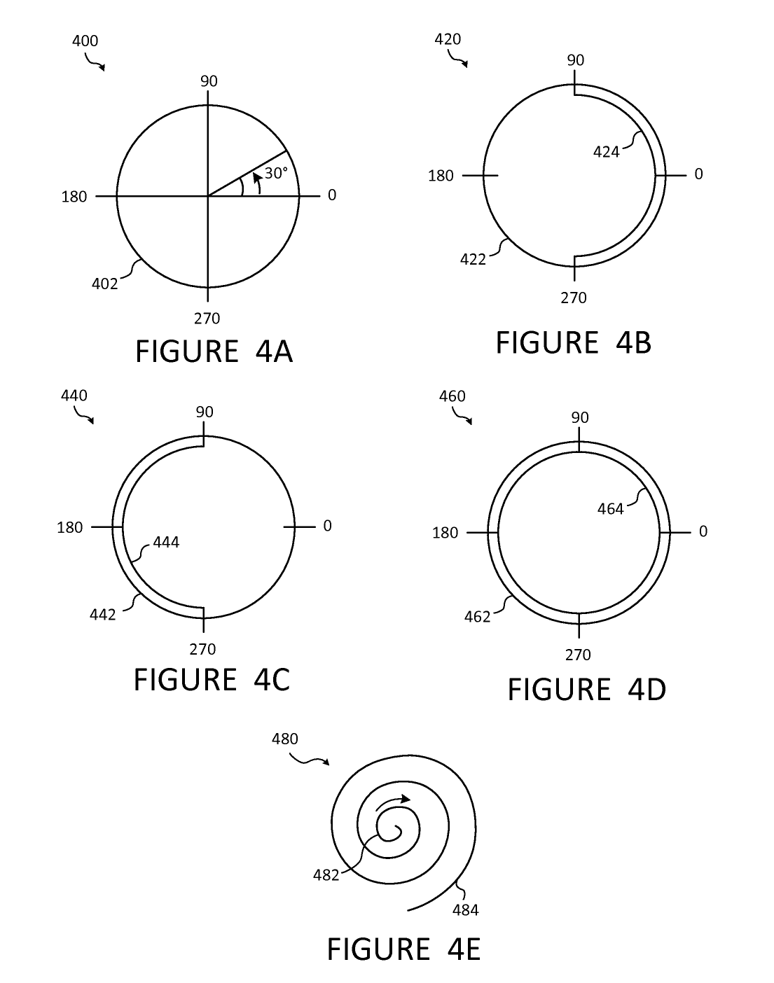

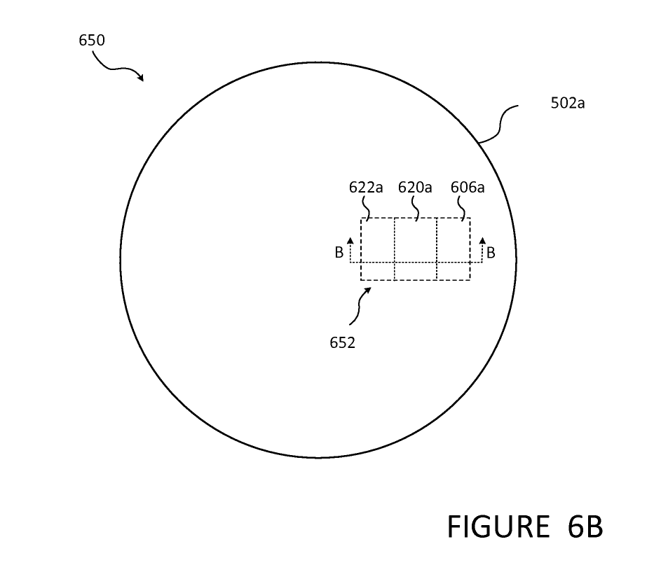

FIG. 4A shows an example software interface diagram configured to be used to specify an angle of corneal incision. Laser machine setup interface 400 includes a reference circle 402 divided into four quadrants. A cut angle of the multiple plane incision is defined for the incision planes, in the form of steps, sloping from the exterior towards anterior of the cornea, as discussed later with respect to FIG. 6A. The cut angle so defined is used during the operation of the laser machine in surgery to make the incision towards the anterior chamber. In various embodiments, the cut angle may be set at 30° with respect to the centerline of the cornea.

FIGS. 4B-4D show examples of software interfaces configured to be used to specify an angular span over which a medical laser machine is active during corneal incision. During cataract surgery (or other types of soft tissue surgery, such as plastic surgery), the laser beam traverses a path, such as a spiral path (as further described with respect to FIG. 4E), multiple times to heat the tissue and cause small subsurface bubbles in the cornea via intrastromal ablation.

FIGS. 4B-4D show various example choices of initially setting up circular paths during which the laser beam is active and is capable of firing pulses for cutting tissue. For example, FIG. 4B shows example setup interface with reference circle 422, and laser beam path 424 extending from 270° to 90°, over which the laser beam is active. Similarly, FIGS. 4C and 4D show example setup interfaces with reference circles 442 and 462, and laser beam paths 444 and 464 extending from 270° to 90°, and 0° back to 0° (whole circle), respectively, over which the laser beam is active. In various embodiments, the reduction of the duty cycle of the laser beam, the portion of the traversal path over which the beam is active, may offer significant performance and speed advantages. As the beam traverses through its spiral cutting path (as described with respect to FIG. 4E below), for each circle within the spiral cutting path, the active portion of the laser beam is defined by the active path set up as above.

In operation, as the laser beam traverses an active path, it may fire a laser pulse according to a software program controlling its firing. The sequence and placement of fired pulses are thus determined under program control and different points on a line of incision may be cut out of sequence depending on the firing program. For example, an incision having endpoints point A and B may be made by pulses starting in the middle of the incision line and progressively moving towards the endpoints A and B by subsequent firings, rather than starting at point A and moving directly to point B.

FIG. 4E shows an example spiral cutting path of a laser beam. In various embodiments, spiral cutting path 480 includes inner circle 482 and outer circle 484. In various embodiments, laser beam may start cutting from inner circle 482, the diameter of which is specified in section 128 of the LSUI (FIG. 1B) as Inner Diameter, and traverse in an spiral fashion outwards to the outer circle 484, the diameter of which is specified in section 128 of the LSUI (FIG. 1B) as Outer Diameter. Several intermediate circles may be completed by the laser beam while traversing from the inner to the outer circle in the spiral. The arrow indicates direction of motion of the laser beam from the inner circle to the outer circle of the spiral.

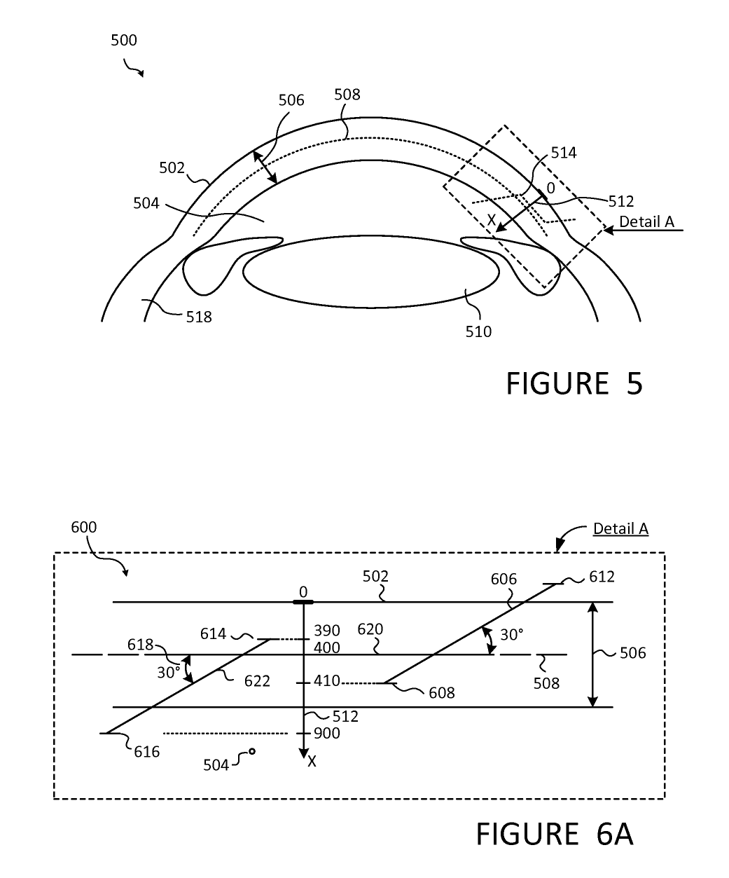

FIG. 5 is an example diagram of a cornea of a human eye showing a location of a corneal incision. Human eye anatomy 500 includes cornea 502, anterior corneal chamber 504, corneal wall thickness or depth 506, lens 510, and sclera 518. Reference frame 512, from distance 0 to X, is used to define the shape of the multi plane incision in the corneal wall. Centerline 508 defines the center of the thickness of the corneal wall, which is located at half the distance of corneal wall thickness measured from the exterior surface of the cornea. Detail A of FIG. 5, including reference frame 512, may be used to define the multi plane incision 514 of the cornea for cataract surgery.

FIG. 6A is an example diagram of a Detail A of the cornea of FIG. 5 showing a detailed structure of an incision in the cornea. FIG. 6A depicts the side view or profile of a multi-plane incision 600 appearing as a “stair-case” looking line. Those skilled in the art will appreciate that the corresponding top view of FIG. 6A, as shown in FIG. 6B below, reveals cut planes as planar surfaces co-planar with each corresponding line section in the stair-case. Thus, the lines shown constitute a longitudinal cross section of the cut planes. A straight cut through the cornea from the exterior surface to the anterior chamber is often associated with leaky incisions, long patient recovery, and post operation medical issues, such as infections, discomfort, and the like. The multi plane incision, including several distinct cut sections, substantially improves these post operative issues by enabling tight incision closure along a longer incision line, quicker patient recovery and tissue healing, and less discomfort.

In various embodiments, corneal wall 502 has centerline 508 and thickness 506. Reference frame 512 extends from 0 at the exterior of the corneal wall to distance X. In various embodiments, geometrically, a first cut section or plane 606 of the multi plane incision has one end at a first or a starting point 612 of the multi plane incision at the exterior surface of the cornea extends inwards towards anterior chamber 504 at a cut angle, for example 30°, with respect to the corneal centerline, as shown, and has a second or an ending point 608 slightly below the corneal centerline. Those skilled in the art will appreciate that the above description of the cut is related to the geometry of the cut and not necessarily how the cut is made with respect to a starting or ending point of the cut. As such, the starting point 612, or the other end of the multi plane incision 616, is not necessarily where the cut is started chronologically. In various embodiments, the actual cut is made using a computer controlled laser beam starting at an interior point of the cornea and spiraling outwards to make the actual incision, as described with respect to FIG. 4E.

The first cut section of the incision corresponds to the anterior side cut discussed in section 140 of the LSUI of FIG. 1B. As an illustrative example, if the corneal wall thickness 506 is 800 microns, then the centerline is at a depth of 400 measured from the 0 point of reference frame 512. All other measurements are also relative to the 0 point of reference frame 512. In this example, the anterior side cut terminates at point 608, for example, at 410 microns below the exterior surface of the cornea.

In various embodiments, a lamellar cut section 620, possibly coincident with the center line, may be made which constitutes a second cut section or a second plane of the multi plane incision. This cut section starts substantially at the end point of the first cut section, the anterior side cut, and extends for some distance where a third cut section or plane 622 of the multi plane incision starts at 614, slightly above the centerline, for example, at 390 microns in the present example. The third section, corresponding to the posterior side cut discussed above, which may be set up using section 136 of the LSUI of FIG. 1B, may be cut with the laser starting from interior and proceeding towards exterior of the cornea. The third section thus extends towards the anterior chamber 504 from point 614, at a cut angle 618, for example 30°, with respect to the corneal centerline, as shown, where it terminates at point 616, for example, at 900 microns, which is in excess of the thickness of the cornea. Thus, the multi plane incision is completed and physically connects the exterior of the cornea to the anterior chamber. The multi plane incision effectively creates a channel from outside of the cornea to the inside to allow surgical instruments to access the lens of the eye. The multi plane incision, thus, resembles a stair-case, with two “downward” sections, the anterior and posterior cuts, and one “horizontal” section, the lamellar cut parallel or coincidental with the corneal centerline.

It is evident that the shortest distance between two points is a straight line and any angle or curvature in the line will serve to increase the length of the line between the same two points. Accordingly, in a multi plane incision in which different cut sections have an angle with respect to other sections, a longer line is created providing better adhesion during healing. Additionally, an incision or cut in a tissue creates two parallel edges around the cut area (tissue separated by the cut). The cut angles serve to create a complementary locking arrangement between the two parallel edges of the incision when they come together, further strengthening the bonding or coupling of the edges of the incision. For example, in a stair-case type incision, as depicted in FIG. 6A, the convex section created above centerline 508, between cut lines 606 and 508 (superimposed on centerline 508), complements the concave section created below centerline 508 by the same cut lines. So, when the convex and the concave sections come together after an operation, they act as a matching lock and key, interlocking teeth, or parts of a jigsaw puzzle, substantially reducing or eliminating a slippage of the cut edges with respect to each other. When such slippage is avoided, healing takes place faster. In contrast, in a straight line cut, the two edges of the incision around the cut may move more easily with respect to each other, hence delaying healing.

The wound architecture in an incision made with a physical blade is different from that of a bladeless incision made with a computer controlled laser. Implementing a stair-case cut with a blade is at best difficult and imprecise because the cut angles 618 of FIG. 6A are substantially impossible to be created without automated laser precision. Additionally, the edges of the incision may be more jagged when made using a blade compared with a laser beam, further delaying the smooth closure of the incision and the healing of the corneal tissue.

Those skilled in the art will appreciate that the stair-case multi plane incision may include more “steps,” meaning having more than two downward sections and more than one horizontal section, thus further lengthening the incision, without departing from the spirit of the present disclosures.

In various embodiments, the multi plane incision is made based on the calculations described with respect to equations (1)-(9) above and more specifically based on the user input and results generated by the LSUI of FIG. 1B, using such equations.

Ring Lamellar Cut parameters shown in FIG. 1B are determined at least using equations (1)-(4) and correspond to the Lamerllar cut 620 aligned with centerline 508 at a depth of approximately half the limbal pachy of FIG. 6A. With continued reference to FIG. 6A, equations (1)-(9), and FIG. 1B, in Ring Lamellar Cut section 128, Limbal Pachy is the thickness of cornea 610, usually provided as an input to LSUI, and Depth in Cornea (in FIG. 1B) is the same as Cut Depth in equation (2) and is the distance from exterior surface of cornea 502 to centerline 508, for example, half of limbal pachy. The Inner and Outer Diameters are the settings for the inner and outer diameters of the smallest (482) and largest (484) circles in the spiral cut path shown in FIG. 4E, determined using at least equations (3) and (4). Energy refers to the energy level or beam intensity setting of the laser machine used in surgery.

Anterior Side Cut parameters shown in FIG. 1B are determined at least using equations (5) and (6) and correspond to the angled side cut 606 of FIG. 6A. With continued reference to FIG. 6A, equations (1)-(9), and FIG. 1B, in the Anterior Side Cut section 140, Posterior Depth corresponds to how deep cut plane 606 goes into the layers of the cornea. Diameter is the same as Anterior Side Cut Diameter and does not change. Cut Position 1 and Cut Position 2 may refer to the location around the eye on the cornea where the cuts are made, according to the desired location of the incision but may vary between right and left eyes and according to the surgeon's choice and discretion, for each of two incisions, if more than one is needed or necessary, to access the lens of the eye. Cut Angle 1 and Cut Angle 2 signify the angle of the cut plane 606 in each of the separate incisions, corresponding to Cut Positions 1 and 2, respectively. Energy refers to the energy level or beam intensity setting of the laser machine used in surgery.

Posterior Side Cut parameters shown in FIG. 1B are determined at least using equations (7)-(9) and correspond to the angled side cut 622 of FIG. 6A. With continued reference to FIG. 6A, equations (1)-(9), and FIG. 1B, in the Posterior Side Cut section 136, Anterior Depth is the starting point of side cut plane 622 corresponding to the depth of the point 614 and Posterior Depth is the ending point of side cut plane 622 corresponding to the depth of the point 616, and Diameter is the Inner Diameter plus a constant and determines the point at which this section of the incision is made by the laser beam as it travels on its spiral path. Energy refers to the energy level or beam intensity setting of the laser machine used in surgery. Side Cut Angle is the angle of cut plane 622 with respect to centerline 508, as shown, and may generally be the same as the Cut Angle in the Anterior Side Cut.

FIG. 6B is an example diagram of the Detail A of the cornea of FIG. 5 showing the top view corresponding to the side view shown in FIG. 6A of the incision at the limbus of the cornea. Side view 650 includes top view of example multi-plane incision 652 having three planar sections 606a, 620a, and 622a, corresponding to cut sections 606, 620, and 622 of FIG. 6A. Similarly, cut sections 606, 508, and 622 correspond with cross section B-B of multi-plane incision 652.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

1. A method of making an incision using a laser machine, the method comprising:
obtaining a measurement of a thickness of a tissue to be cut, wherein a cut in the tissue is part of a multi plane incision;
using a set of equations to calculate setup parameters of the laser machine based on the measurement of the thickness of the tissue, wherein the setup parameters allow the laser machine to make the multi plane incision; and
setting up the laser machine using the calculated setup parameters.

2. The method of claim 1, further comprising using a computer laser setup user interface to enter the measurement.

3. The method of claim 1, further comprising using a computer laser setup user interface to enter the measurement and to view the calculated setup parameters.

4. The method of claim 1, further comprising using the laser machine to perform surgery.

5. The method of claim 1, wherein the laser machine set up with the calculated setup parameters is used in cataract surgery.

6. The method of claim 1, wherein the cut is at least one of a lamellar cut, an anterior cut, and a posterior cut of the cornea of human eye in cataract surgery.

7. The method of claim 1, wherein the set of equations comprises at least some of the following equations:

| | |
|---|---|
| (a) | Anterior Side Cut Change = Limbal Pachy / (Tangent (Cut Angle)); |
| (b) | Cut Depth (in cornea) = Limbal Pachy × A; |
| (c) | Outer Diameter = (Anterior Side Cut Diameter − (B × Anterior |
| | Side Cut Change)) + C; |
| (d) | Inner Diameter = (Outer Diameter − (B × Shelf Length)) − D; |
| (e) | Posterior Depth = (Limbal Pachy × A) + E; |
| (f) | Cut Depth (in cornea) = Limbal Pachy × A; |
| (g) | Anterior Depth = (Limbal Pachy × A) − E; |
| (h) | Posterior Depth = (Limbal Pachy + F); |
| (i) | Diameter = (Inner Diameter + D); |

Wherein A, B, C, D, E, F, Anterior Side Cut Diameter, and Cut Angle are constants, and Limbal Pachy is a measured thickness of cornea.

8. The method of claim 1, wherein the multi plane incision includes at least three cuts wherein adjacent cuts are not aligned.

9. The method of claim 1, wherein the multi plane incision has the form of a stair-case.

10. A Laser Setup User Interface (LSUI) comprising:
an input software module configured to receive input from a user;
a processing software module configured to calculate setup parameters for a laser machine based on the input from the user and based on a set of equations; and
a display software module configured to display the calculated setup parameters.

11. The LSUI of claim 10, further comprising a reticle configured to be used to aim a laser beam of the laser machine.

12. The LSUI of claim 10, further comprising multiple display sections, each display section configured to display a subset of the setup parameters for the laser machine usable to make a different cut section, corresponding to the subset of the setup parameters, of a multi plane incision.

13. The LSUI of claim 10, wherein the input from the user comprises a thickness of a cornea of a patient.

14. The LSUI of claim 10, wherein the set of equations comprises at least some of the following equations:

| | |
|---|---|
| (a) | Anterior Side Cut Change = Limbal Pachy / (Tangent (Cut Angle)); |
| (b) | Cut Depth (in cornea) = Limbal Pachy × A; |
| (c) | Outer Diameter = (Anterior Side Cut Diameter − (B × Anterior |
| | Side Cut Change)) + C; |
| (d) | Inner Diameter = (Outer Diameter − (B × Shelf Length)) − D; |
| (e) | Posterior Depth = (Limbal Pachy × A) + E; |
| (f) | Cut Depth (in cornea) = Limbal Pachy × A; |
| (g) | Anterior Depth = (Limbal Pachy × A) − E; |
| (h) | Posterior Depth = (Limbal Pachy + F); |
| (i) | Diameter = (Inner Diameter + D); |

Wherein A, B, C, D, E, F, Anterior Side Cut Diameter, and Cut Angle are constants, and Limbal Pachy is a measured thickness of cornea.

15. The LSUI of claim 10, wherein the LSUI is configured to be used to set up the laser machine for cataract surgery.

16. The LSUI of claim 10, wherein the laser machine is a femtosecond laser system.

17. A method of laser cataract surgery, the method comprising:
obtaining a measurement of a thickness of a patient's cornea to be cut for removing a lens with cataract, wherein the cut is a multi plane cut;
using a set of equations to calculate setup parameters of a laser machine based on the measurement of the thickness of the cornea, wherein the setup parameters allow the laser machine to make the multi plane cut through the cornea to allow surgical instruments to access the lens; and
setting up the laser machine using the calculated setup parameters.

18. The method of claim 17, further comprising performing a cataract surgery using the laser machine set up with the calculated setup parameters.

19. The method of claim 17, wherein the laser machine is a femtosecond laser system.

20. The method of claim 17, wherein the set of equations used to calculate the setup parameters is used by a Laser Setup User Interface (LSUI) software, which receives the measurement of thickness of the patient's cornea as input.
